(19) United States
(12) Patent Application Publication
(10) Pub. No.: US 2021/0304867 A1
(43) Pub. Date: Sep. 30, 2021

(54) APPLICATION WITH BEHAVIOR CHANGE USER INTERFACE RESPONSES

(71) Applicant: MindSciences, INC., Worcester, MA (US)

(72) Inventors: Judson Brewer, Worcester, MA (US); Mark Mitchnick, East Hampton, NY (US); Joshua Roman, Scituate, MA (US)

(21) Appl. No.: 16/831,996

(22) Filed: Mar. 27, 2020

(51) Int. Cl. G16H 20/60; G16H 50/30; G06F 3/01; G06F 3/0482

(52) U.S. Cl. CPC G16H 20/60; G06F 3/0482; G06F 3/015; G16H 50/30

(57) ABSTRACT

The present technology pertains an application configured to instruct a user to think about an aspect of a craving to partake in an undesired behavior, and after the presentation of the information presenting a user interface instructing the user to think about the aspect of the craving to partake in the undesired behavior. The user interface of the application can present an interface that is configured to receive a craving score that is an indication of how the craving to partake in the undesired behavior has changed relative to a time prior to the presentation of the information instructing the user to think about the aspect of the craving to partake in the undesired behavior. The present technology can proactively adjust to help a user to take helpful actions towards behavior change.

### TECHNICAL FIELD

The disclosed technology pertains to a software application that encourages behavior change through user interface responses, and more specifically to a software application that includes a sequence of user interfaces that receive responses that encourage behavior change, track behavior change, and dynamically recommend actions to encourage behavior change.

### BACKGROUND

Individuals are constantly looking for ways to break bad habits: skipping a smoke break, stopping after one or two drinks, eating less, or refraining from unhealthy foods. However, there is no systematized way for individuals to change these habits using technology that fosters mindful or thoughtful contemplation on the habits themselves.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 illustrates an example configuration of devices and a network in accordance with some aspects of the present technology;

FIGS. 2A, 2B, 2C, 2D, 2E, and 2F illustrate an example user interface in accordance with some aspects of the present technology;

FIGS. 3A and 3B illustrate an example method in accordance with some aspects of the present technology; and

FIG. 4 illustrates an example system embodiment in accordance with some aspects of the present technology.

### DESCRIPTION OF EXAMPLE EMBODIMENTS

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

The present technology addresses the need in the art for a technology that facilitates mindful contemplation and engagement in an undesirable activity, with the purpose of ultimately enabling someone to refrain from that undesirable activity. In recent years, substantial cultural attention has been directed to the practice of mindfulness: “the psychological process of purposely bringing one's attention to experiences occurring in the present moment without judgment.” While originating in Buddhist meditative practices, mindfulness has developed into a secular phenomenon. Books, workshops, and mobile apps facilitating mindfulness meditation have flooded the marketplace, reflecting a growing consumer demand for mindfulness, or for the benefits of the practice.

Regarding those benefits, mindfulness has subsequently become the subject of scientific inquiry. Studies have revealed the utility of mindfulness as a treatment for mental illness, including depression and anxiety, along with other health issues, including pain management. One such mental illness for which mindfulness shows promise is substance-abuse disorders.

The vices of today's society are plentiful, and every day people look to take steps to end undesirable behaviors: overeating, drinking, smoking, drug abuse, and countless other habits hold sway over people's lives to a high degree. With the advent of mindfulness research in this space and the clear market for mindfulness-facilitating products, there is a clear need in the art for a technology which facilitates mindfulness-based behavior-changing techniques.

The present technology fills this need. The present technology provides a sequence of dependent user interfaces that receive responses that encourage behavior change, track behavior change, and dynamically recommend actions to encourage behavior change. The technology provides a user interface to guide a user through mindfully partaking in an undesirable activity, to record data regarding a user's thoughts pertaining to the activity, and to suggest actions. The user experiences a prompt to contemplate the undesirable activity, and to assess a craving level for that undesirable activity after going through the contemplation

The present technology provides an interface to bridge a gap between a computer and human cognition. As anyone that has tried to engage in self-improvement activities understands, whether a new year's resolution, a diet, or breaking a habitual undesired behavior, the human brain functions to resist these objectives. A person can consciously know that a behavior is bad for them, but will find it difficult to resist that behavior because merely knowing something is bad is not the same as resetting the brains reward value associated with the behavior. A reward value is a value that the brain associates with a behavior. For example, if the brain has a strong reward value associated with smoking, then the brain will cause a person to crave a cigarette—even if the person knows that eating smoking is not good for them. The present technology can modify the reward value stored in the brain associated with an undesired behavior.

In some embodiments, a reward value can be determined according to the following model, called the Rescorla-Wagner reinforcement learning model:

Vₜ₊₁=Vₜ+αδt

The Rescorla-Wagner reinforcement learning model posits that a future reward value (Vₜ₊₁) of a given behavior is dependent upon its previous reward value (Vₜ)+a learning signal (αδt). The learning signal is dependent upon what's called a prediction error (δt), which is a discrepancy between an actual outcome of the behavior and what is expected. α is a static subject-level parameter (a constant).

Even when a person is instructed in techniques that facilitate behavior change in a way that circumnavigates the brains natural tendency to retain a habit, behavior change is still difficult without external help. The present technology provides that external help by combining information about techniques that facilitate behavior change with an interface that can be used to record data points that can be used measure progress towards the desired behavior change. The present technology can utilize this data to show a user how un-rewarding the undesired behavior is which will change the reward value stored in their brain. When a behavior is seen a less rewarding by the brain, the person experiences less of a desire to engage in the behavior. Accordingly the present technology provides an application that collects data and utilizes the data to cause a change in a reward value perceived by the brain associated with the undesired behavior.

The present technology can utilize this data to adjust aspects of the guidance provided to the user to individualize the activities taken by a user to lead to faster and more lasting behavior change. The present technology can also provide timely notifications and alerts when the user might be tempted to resort to the brain's natural tendency towards engaging in habitual behavior.

The present technology includes instructions stored on a non-transitory computer-readable medium. When executed by a processor, these instructions cause the processor to present information instructing a user to think about an aspect of a craving to partake in an undesired behavior. After the presentation of this information, the processor presents an interface that is configured to receive a craving score. This craving score is an indication of how the craving to partake in the undesired behavior has changed since the information instructing a user to think about an aspect of a craving to partake in an undesired behavior was presented. Finally, the processor receives the craving score in the user interface.

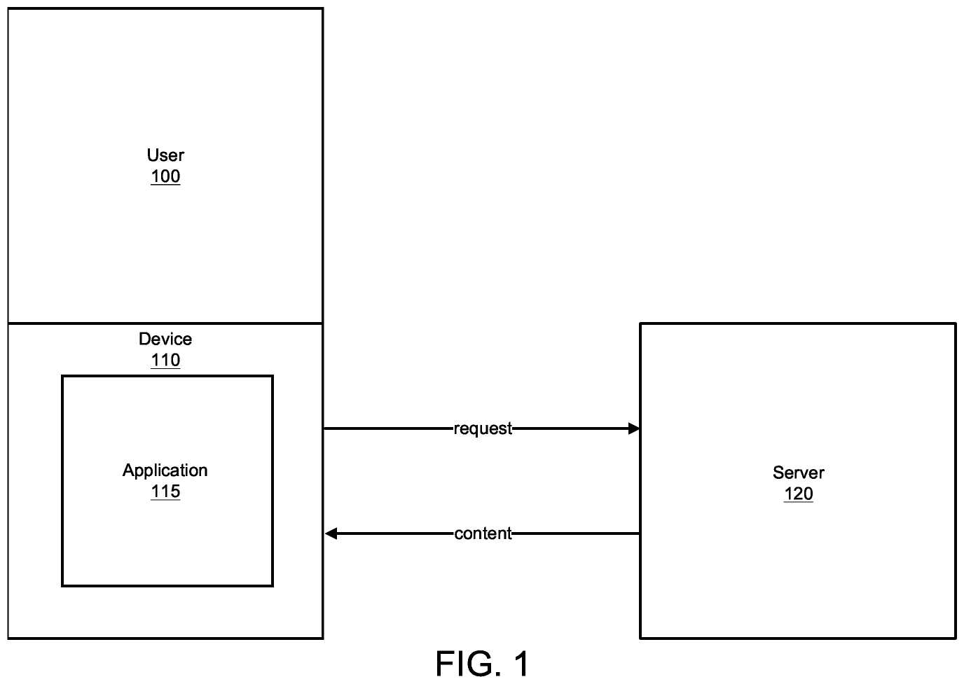

FIG. 1 illustrates an example network environment for providing access to user interface 200 in accordance with some aspects of the present technology. Although FIG. 1 illustrates a client server network environment 100, other embodiments of the subject technology may include other configurations.

User 100 can operate device 110 to interact with application 115. User 100 can be any individual interested in changing an aspect of their behavior. This can include reducing a habit such as smoking, drinking, excessive eating, or another vice, or modifying another aspect of behavior, such as waking up earlier or going to the gym with more frequency. Device 110 can be any computing device capable of executing application 115. It can be a mobile device, tablet, laptop, home computer, wearable device, or other computing device. In some embodiments, device 110 can receive biometric data measured from user 110, such as brain activity, heart rate, body temperature, breathing rate, or skin conductance using one or more sensors on device 110 or another device in communication with device 110. For example, a smart watch might both execute application 115 and include one or more biometric sensors, whereas a laptop might only execute application 115 and receive biometric data from another device having the necessary sensors to measure the biometric data.

Application 115 can provide an interface to bridge a gap between a computer and human cognition. Application 115 can provide interfaces that provide information about techniques that facilitate behavior change and can provide interfaces that can be used to record data points that can be used to change the reward value associated with the behavior in a user's brain. In some embodiments, the present technology can be used measure progress towards the desired behavior change. Application 115 can utilize this data to show a user that they are making progress, thus reinforcing their gains towards the behavior change, and motivating the user to keep with the activities leading towards the behavior change. Application 115 can utilize this data to adjust aspects of the guidance provided to the user to individualize the activities taken by a user to lead to faster and more lasting behavior change. Application 115 can also provide timely notifications and alerts when the user might be tempted to resort to the brain's natural tendency towards engaging in habitual behavior.

In some embodiments, application 115 can cause device 110 to send a content request to server 120. This request asks for content with which to populate one or more user interfaces of application 115. For example, server 120 can provide user profile and account information, updated user interfaces, individualized activities, etc. Server 120 can fulfill the request by serving content back to device 110. The content can vary by the behavior which user 100 is seeking to modify.

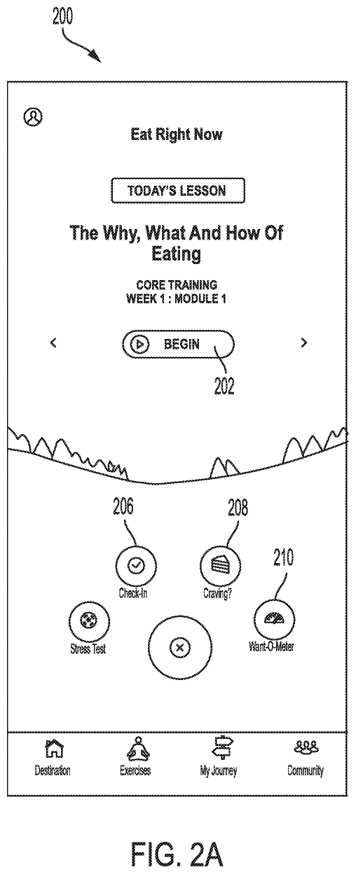

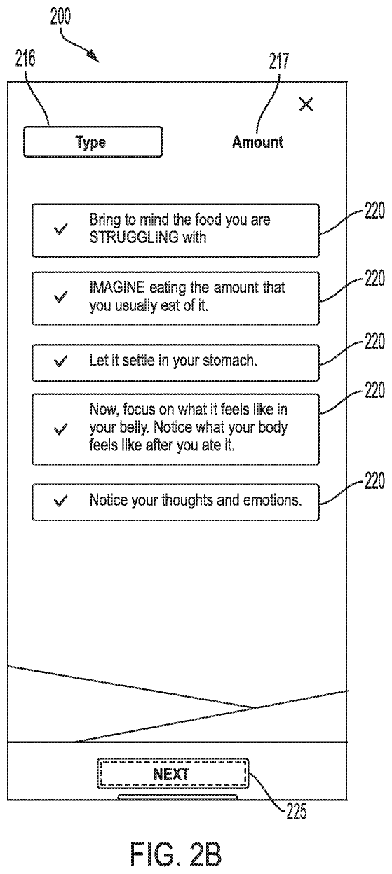

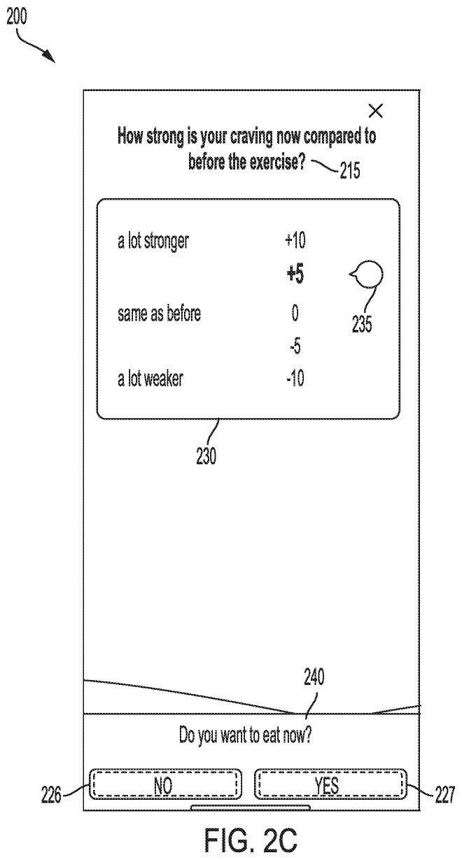

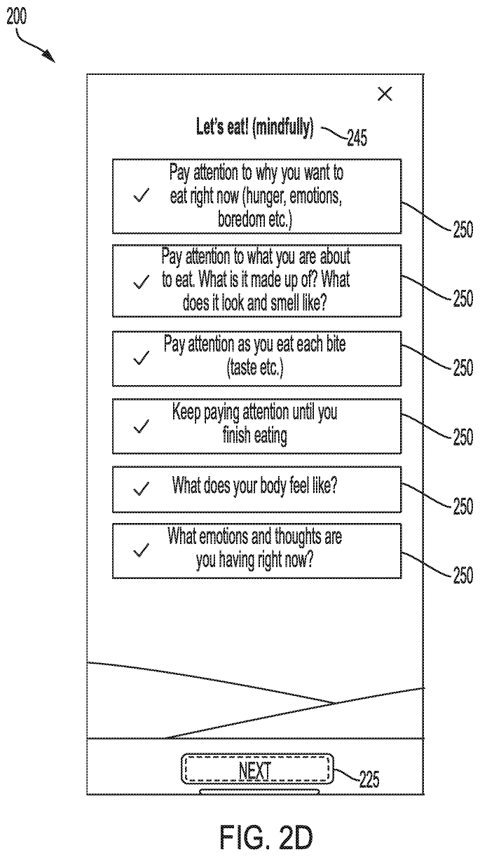

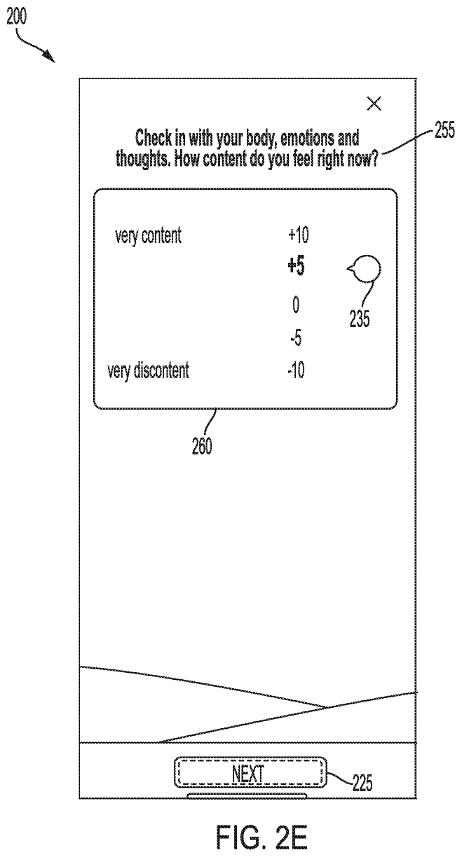

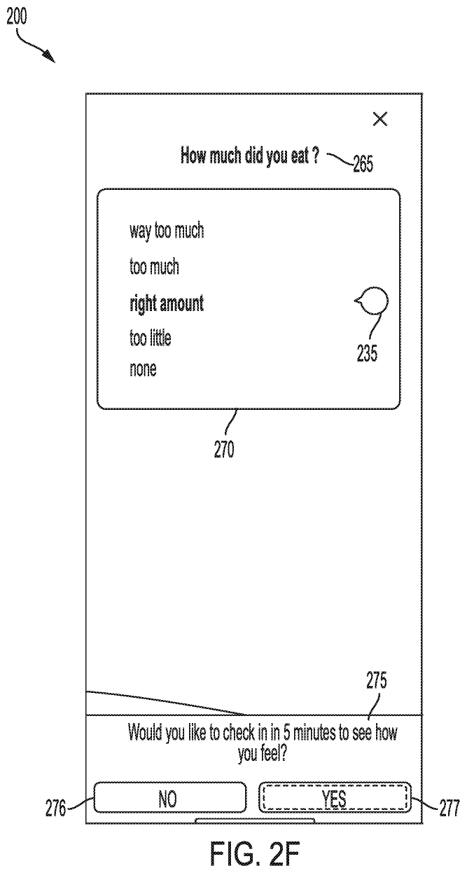

In some embodiments, server 120 can store past data of user 100, including data entered into application 115 or data collected by device 110, such as biometric data. This data can be used to identify outliers in craving scores and contentedness scores (further explained in FIGS. 2A-2F), to actively suggest content such as activities that could reduce cravings, to predict when cravings might occur based on biometric and user input data, or for other purposes.

FIGS. 2A-2F illustrate an example user interface 200 that can be presented by application 115, in accordance with some aspects of the present technology. User interface 200 can progress through the screens presented in FIGS. 2A-2F.

FIG. 2A shows a first screen of user interface 200. A user 100 has various options presented. Selecting button 202 can trigger application 115 to begin a training module related to reducing an undesired behavior, in this case over-eating. Selecting button 206 can trigger application 115 to begin a check-in on the progress of user 100 towards reducing over-eating. Selecting button 208 can trigger application 115 to initiate a user interface-facilitated experience of mindful contemplation and consumption related to over-eating. Selecting button 210 can trigger application 115 to present data and other measurements related to past cravings. In some embodiments, particularly those targeting addictive behaviors, there may be a button which can enable user 100 to call a sponsor.

FIG. 2B presents a progression of user interface 200 that is presented in response to application 115 receiving a selection of button 208 in FIG. 2A, corresponding to a craving. Craving category selectors 216 and 217 present user 100 with options to choose whether the craving relates to a certain type of food or a quantity of food, respectively. As shown in FIG. 2B, application 115 has received a selection of craving category 216. In some embodiments, user interface 200 may appear without prompting by user 100, such as when a craving is predicted by biometric or user input data. This data can be historical data provided by server 120, or data received by device 110 that triggers application 115 to prompt user 100 with user interface 200 as shown in FIG. 2B.

FIG. 2B further shows a collection of mindful contemplation instructions 220 relating to mindful contemplation regarding the undesired behavior. The present technology appreciates that when a user thinks mindfully about a habitual behavior, different mental pathways are engaged in the brain than the typical pathways utilized by the brain when a craving to engage in a habitual behavior occurs. A typical pathway utilized by the brain reinforces habitual behavior, but when a user thinks more about the behavior, the person gains more agency over the behavior and how it makes them feel and the reward value perceived by the user from engaging in the behavior. Accordingly, application 115 presents a collection of mindful contemplation instructions 220 that are designed to encourage the user to approach the habitual behavior mindfully and thereby trigger a more beneficial mental pathway by accessing an accurate reward value that is stored in their brain, rather than habitually and mindlessly acting on a non-descript craving trigger. This contemplation helps to bring into conscious awareness not only previously stored reward values from the behavior, but brings these together with current physiological and emotional states (e.g. level of hunger, amount of stress). This contemplation, in turn leads to an accurate mental appraisal of whether the habitual behavior is indeed desirable. Application 115 is thus a tool that triggers non-typical, and more constructive, mental pathways in the user 100 for approaching the habitual behavior. In some embodiments, application 115 can highlight each instruction in the collection of mindful contemplation instructions 220 for a set period of time, before the next mindful contemplation instruction 220 is highlighted, and so on in order to emphasize the importance of the individual statements and/or the sequence of contemplation.

After application 115 has concluded its presentation of mindful contemplation instructions 220, user 100 can select continuation prompt 225. When user 100 selects continuation prompt 225, this selection can trigger application 115 to move to the next portion of the user experience, as shown in FIG. 2C.

FIG. 2C presents the progression of user interface 200 that is presented in response to application 115 receiving a selection of continuation prompt 225 in FIG. 2B. As shown in FIG. 2C, application 115 presents user interface 200 that is configured to receive a craving score. This craving score is an indication of how the craving to partake in the undesired behavior has changed relative to a time prior to the presentation of the information instructing the user to think about the aspects of the craving, as embodied in instructions 220 illustrated in FIG. 2B. Instructions 220 cause a user to contemplate a current reward value associated with the undesired behavior in the user's current physiological state and the user's current emotional state. FIG. 2C invites a user to provide an indication of whether their craving state is stronger (e.g. very rewarding) or weaker (e.g. not rewarding) after they have followed instructions 220. The data that the user provides in user interface 200 shown in FIG. 2C can be considered a proxy for the user's current reward value associated with the behavior.

FIG. 2C shows prompt 215 asking user 100 to compare their current intensity for the craving to the intensity of the craving at a point in time before the exercise presented in FIG. 2B. User input prompt 230 can receive a score from user 100 that indicates the relative strength of the current craving compared to the pre-exercise craving strength. User interface 200 provides a data entry interface to receive the craving score. As illustrated in FIG. 2C, user interface 200 provides slider 235 as the data entry interface that can be adjusted by user 100 to match the appropriate place on the scale. While slider 235 is shown, it should be appreciated that this is just an example and any other suitable user interface element that can receive data indicating a craving score can be used.

As addressed above, the present technology provides an interface to bridge a gap between a computer and human cognition so that application 115 can provide external help to user 100 trying to modify a habitual behavior and functions to facilitate behavior change. Even if a user were to mindfully engage in a habitual activity without the help of application 115, which is helpful, the user would not fully recognize the benefit of the activity without recording a craving score as provided in FIG. 2C. For example, a user might mindfully contemplate a habitual activity prior to engaging in the habitual activity and as a result the user might feel less inclined to engage in the habitual activity, but the user might not bring that change in inclination into their conscious mind. Additionally, without the sequence of interfaces shown in FIG. 2B and then FIG. 2C that first causes a user to engage in the mindful contemplation of the undesired behavior followed by the receipt and viewing of the craving score, the combination of a reward value associated with the undesired behavior, the user's physiological state, and the user's emotional state would not be realized and concretized for immediate feedback by the user. This immediate feedback results in an effect on the user's subsequent action with respect to the undesired behavior. The user interface illustrated in FIG. 2C is a tool to force the user to recognize that their mindful activity is working—which is a reward, and the brain responds strongly to rewards (reward-based learning, a type of learning that happens through “feedback” in terms of how rewarding or unrewarding a behavior is, is the strongest learning mechanism currently known in science, winning Eric Kandel the Nobel Prize in the year 2000).

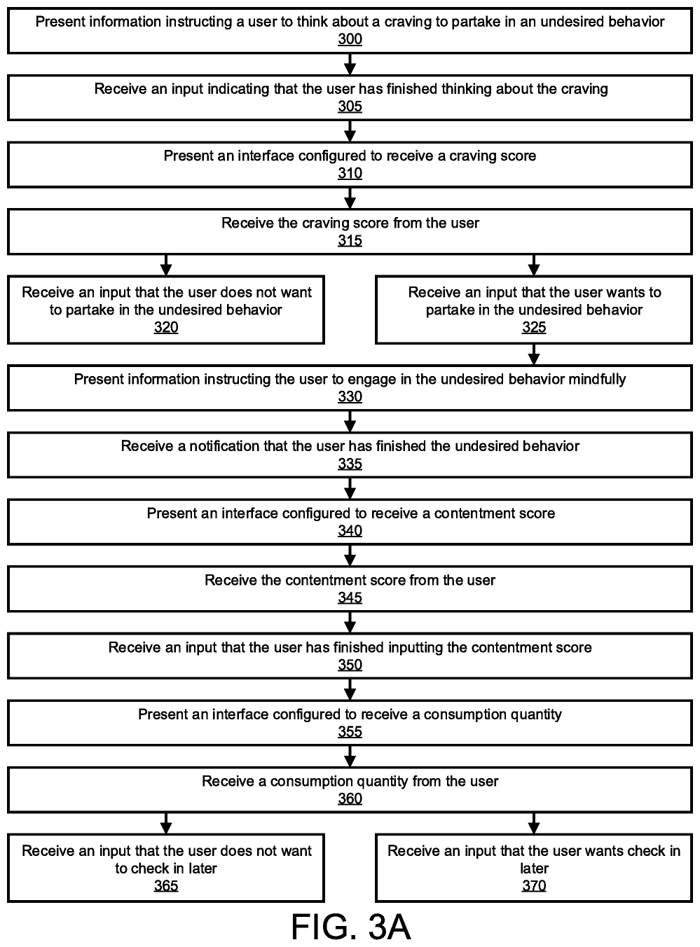

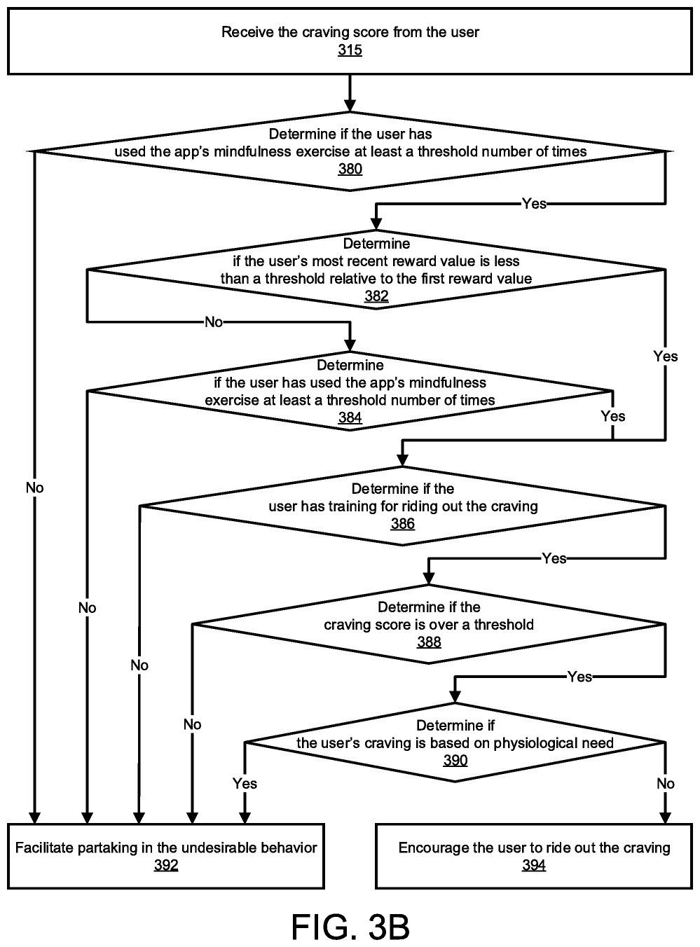

In some embodiments, the value reflecting the change in the user's craving intensity can be used to predict whether engaging in the activity is expected to reduce the user's reward value tied to the undesired behavior. A reduction in the craving of the intensity can signal that a user has already experienced a negative value for the learning signal (αδt) in the Rescorla-Wagner reinforcement learning model addressed above. Further, and as addressed in more detail below with respect to FIG. 3B the present technology can predict whether mindful contemplation while engaging in the behavior would continue to reinforce the negative learning that would result in a perception in a decreased reward value (Vₜ₊₁). For example, as noted, the craving score input in FIG. 2C can be a proxy for a current reward value (Vₜ) associated with that behavior. To predict how the current reward value would change after mindfully engaging in the behavior, application 115 can predict a future reward value (Vₜ₊₁) by estimating an expected learning signal (αδt), which can be approximated from data derived from a population of users that have used application 115 to modify similar behaviors in similar circumstances.

The benefits of collecting data on user craving scores goes beyond in-the-moment mental nudges that application 115 encourages a user to take part in. Application 115 can generate graphic visualizations of past user craving scores can be presented to show the progress of a user over time, encouraging the user to stay on a successful trajectory. Data stores of craving scores can be used to predict future cravings based on time of day, biometric readings, or other factors. These predictions, generated by application 115, can then be used as factors to better encourage certain behaviors in user 100 to ultimately lead to a more successful behavioral change outcome. Application 115 can incentivize user 100 with rewards, such as in-app badges or tokens, based on the trajectory of craving scores through time, or refraining from the undesired behavior despite a high craving. In some embodiments, these badges or tokens can have a social aspect, where other users of application 115 can see the progress and achievements of others.

Sometimes the user might actually crave the habitual activity more than before the mindfulness exercise (e.g. when hungry), but in general, and through practice, the user should see their cravings reduce. It has been found that, using the Rescorla-Wagner model, reward values associated with undesired behaviors can drop to near zero after using the present technology 10-15 times.

Accordingly, the present technology is a tool that not only triggers a particular mental pathway that gives a user more agency over a habitual activity, but triggers a recognition of the benefit of their effort, and can result in the immediate perception of that benefit. The perception of a benefit can be interpreted by the brain as a reward that, at least, can reinforce the need and desire to mindfully engage in a habitual activity instead of bypassing the mindfulness activity to engage in the habitual activity without delay. As such the present technology causes benefits that are not achieved without the external assistance provided by application 115.

Application 115 presents a continuation prompt 240 which can encourage user 100 to proceed in one of two ways. The selection of continuation sub-prompt 226 can trigger application 115 to instruct user interface 200 to present the screen shown in FIG. 2A. This corresponds to user 100 acting on an accurately perceived combined reward value that has lessened the craving to partake in the undesired behavior to the point where the original craving is not strong enough to drive the behavior. Alternatively, the selection of continuation sub-prompt 227 can trigger application 115 to instruct user interface 200 to present the screen shown in FIG. 2D. This corresponds to encouraging user 100 to mindfully partake in the undesired behavior. User 100 can choose between the two options.

In some embodiments, application 115 can actively encourage user 100 to choose continuation sub-prompt 226 or continuation sub-prompt 227 based on internal predictions (i.e. combined reward value) of which option will better discourage the undesirable behavior in the long run (see FIG. 3B for more detail). These predictions can be generated by past behavior of user 100, past biometric data, current biometric data, or other factors. Encouragement can take the form of displaying data from user 100, highlighting the choices, changing the color of the choices, changing the size of the choices, animating the choices, or using other visual means to nudge user 100 to one of continuation sub-prompts 226 and 227. In some embodiments, more or fewer options can be available, such as encouraging user 100 to engage in the undesirable activity after a temporal delay.

FIG. 2D presents the progression of user interface 200 which is displayed after user 100 selects continuation sub-prompt 227 in FIG. 2C, indicating a desire to mindfully partake in the undesirable behavior Prompt 245 encourages user 100 to eat in a mindful manner.

FIG. 2D further shows a collection of mindful consumption instructions 250 relating to mindful partaking of the undesired behavior. The present technology appreciates that when a user partakes mindfully in a habitual behavior, different mental pathways are engaged in the brain than the typical pathways utilized by the brain when engagement in that habitual behavior occurs. A typical pathway utilized by the brain reinforces habitual behavior (based on old, cached reward values that don't reflect current physiological and emotional states), but when a user mindfully engages, the person gains more agency over the behavior and how it makes them feel and the reward value perceived by the user from engaging in the behavior (which brings the user's rewards regarding the behavior together with current physiological and emotional states). Accordingly, application 115 presents a collection of instructions 220 that are designed to encourage the user to approach the habitual behavior mindfully and thereby trigger a more beneficial mental pathway through accurate reward valuation. The results of the current application of mindful eating update the cached reward value for future retrieval. For example, paying attention when overeating helps the user see how un-rewarding this behavior is now, compared to the previously stored memories of the activity. Application 115 is thus a tool that triggers non-typical, and more constructive mental pathways in the user 100 for approaching the habitual behavior. In some embodiments, application 115 can highlight each instruction in collection of mindful consumption instructions 250 for a set period of time, before the next mindful consumption instruction 250 is highlighted, and so on in order to emphasize the importance of the individual statements and/or the sequence of contemplation.

After application 115 has concluded its presentation of instructions 220, user 100 can select continuation prompt 225. When user 100 selects continuation prompt 225, this selection can trigger application 115 to move to the next portion of the user experience, as shown in FIG. 2E.

FIG. 2E presents the progression of user interface 200 that is presented in response to application 115 receiving a selection of continuation prompt 225 in FIG. 2D. As shown in FIG. 2E, application 115 presents user interface 200 that is configured to receive a contentedness score, prompted by prompt 255. This contentedness score is an indication of how content user 100 is currently feeling, after mindful partaking of an undesirable behavior. This exercise “forces” the user to accurately perceive how rewarding (or un-rewarding) the behavior was, which leads to updating of the cached reward value in memory. This is a critical component, as users often do not pay attention to the results of behavior, and thus repeat habitual behaviors due to acting out cached reward values from “outdated” memories. User input prompt 260 can take in a score from user 100 that indicates the current contentedness of user 100. User interface 200 provides a data entry interface to receive the contentedness score As illustrated in FIG. 2E, user interface 200 provides slider 235 as the data entry interface that can be adjusted by user 100 to match the appropriate place on the scale. While slider 235 is shown, it should be appreciated that this is just an example and any other suitable user interface element that can receive data indicating a craving score can be used.

In some embodiments, the contentedness score can be a proxy for the value of the learning signal (αδt) in the Rescorla-Wagner reinforcement learning model addressed above. A contentedness score that reflect that the engaging in the undesired behavior did not provide the effect the user was seeking suggests that the learning signal was a negative and thereby results in a decrease in reward value (Vₜ₊₁).

After application 115 has received the contentedness score, user 100 can select continuation prompt 225. When user 100 selects continuation prompt 225, this selection can trigger application 115 to move to the next portion of the user experience, as shown in FIG. 2F.

FIG. 2F presents the progression of user interface 200 that is presented in response to application 115 receiving a selection of continuation prompt 225 in FIG. 2E. Prompt 265 presents a question asking user 100 to report how much was consumed by user 100 (assuming the behavior involves consuming food, drink, or drug, etc.). In some embodiments, wherein the behavior is related to quitting smoking or drinking, this prompt may ask for how long user 100 smoked, how much user 100 drank, or other habit-specific consumption questions. Application 115 presents user interface 200 that is configured to receive a consumption quantity. User input prompt 270 can take in a score from user 100 that indicates the consumption quantity of user 100. User interface 200 provides a data entry interface to receive the consumption quantity. As illustrated in FIG. 2F, user interface 200 provides slider 235 as the data entry interface that can be adjusted by user 100 to match the appropriate place on the scale. While slider 235 is shown, it should be appreciated that this is just an example and any other suitable user interface element that can receive data indicating a consumption quantity can be used.

Application 115 presents a check-in prompt 275 which can encourage user 100 to proceed in one of two ways. The selection of check-in sub-prompt 276 can trigger application 115 to instruct user interface 200 to present the screen shown in FIG. 2A and end the process of mindful contemplation and partaking of an undesirable behavior. The selection of check-in sub-prompt 277 can trigger application 115 to, at a temporal delay, prompt user 100 to complete a check-in regarding how user 100 is feeling. This check-in can involve data collection, walk-throughs of mindful activities, or other prompts. The delay of the check-in and the content of the check-in can be influenced by the past behavior of user 100, the current craving and contentedness scores, biometrics, or other factors. In some embodiments, application 115 can actively encourage user 100 to choose check-in sub-prompt 276 or check-in sub-prompt 277 based on internal predictions of which option will better discourage the undesirable behavior in the long run. These predictions can be generated by past behavior of user 100, past biometric data, current biometric data, or other factors. Encouragement can take the form of displaying data from user 100, highlighting the choices, changing the color of the choices, changing the size of the choices, animating the choices, or using other visual means to nudge user 100 to one of check-in sub-prompts 276 and 277. In some embodiments, more or fewer options can be available, such as encouraging user 100 to engage in the undesirable activity after a temporal delay.

FIG. 3A illustrates an example method in accordance with some aspects of the present technology. Information is presented instructing user 100 to contemplate a food craving, input a craving score into user interface 200, and then either mindfully consume the food or refrain from consumption.

The method begins when application 115 presents (300) information instructing user 100 to think about a craving to partake in an undesirable behavior, as shown in FIG. 2B. Once user 100 has finished thinking about partaking in the craving, application 115 is configured to receive (300) an input indicating that the user has finished thinking and is ready to proceed.

Application 115 proceeds to present (310) an interface configured to receive a craving score, as shown in FIG. 2C. For example, application 110 can receive an input via a user interface component, such as slider 235 that indicates the current level of craving relative to a level of craving prior to the beginning of the method. User 100 can choose to proceed with the undesirable activity or refrain. In some embodiments, application 115 can provide a suggestion to user 100 regarding which option to take. For example, in response to the receiving of the craving score, application 115 can make a determination based on the craving score and a profile of user 100 whether to encourage the user to ride it out and refrain from engaging in the undesirable activity by adjusting the look of options or the presence of options in the user interface. An example method for determining when to encourage the user to ride out a craving is addressed in more detail with respect to FIG. 3B. The user interface is configured to receive (320) an input that user 100 does not want to partake in the undesirable activity from continuation sub-prompt 226 and to receive (325) an input that user 100 does want to partake in the undesirable activity from continuation sub-prompt 227.

If user 100 wants to partake in the undesirable activity, application 115 proceeds to present (330) information instructing user 100 to do so mindfully, as shown in FIG. 2D. Once user 100 has finished partaking in the undesirable behavior mindfully, application 115 is configured to receive (335) an input that the user has finished and is ready to proceed. Application 115 can guide user 100 through the mindful partaking process, as shown in FIG. 2D.

Application 115 proceeds to present (340) an interface configured to receive a contentment score, as shown in FIG. 2E. User 100 can use slider 235 to indicate the current level of contentment. Slider 235 allows application 115 to receive (345) the contentment score from user 100. Once user 100 has entered the contentment score, application 115 is configured to receive (350) an input that the user is ready to proceed.

Application 115 proceeds to present (355) an interface configured to receive a consumption quantity, as shown in FIG. 2F. User 100 can use slider 235 to indicate the level of consumption. Slider 235 allows application 115 to receive (360) the consumption quantity from user 100. User 100 can choose to have a check-in at a later time or not; application 115 is configured to receive (365) an input that user 100 does not want to check in later from check-in sub-prompt 276 and to receive (370) an input that user 100 does want to check in later from check-in sub-prompt 277.

FIG. 3B illustrates an example method in accordance with some aspects of the present technology. As addressed above, in some embodiments, application 115 receives a craving score from user 100, and must decide whether or not to facilitate mindful partaking in the undesirable behavior or whether to encourage user 100 to “ride out” the craving (refrain from partaking in the undesirable behavior). Such encouragement can be provide through queues provided in user interface 200 such as in how user interface options 226 and 227 in FIG. 2B are presented. Application 115 can make this determination when it can predict that a future reward value (Vₜ₊₁) will be less than a current reward value (Vₜ) using the Rescorla-Wagner reinforcement learning model.

The method begins when application 115 receives (315) a craving score from user 100. Application 115 can use data from previous mindfulness exercises undertaken by user 100, data from a population of users of application 115, current and past biometric data, or other inputs to decide whether it would be more beneficial for user 100 to mindfully engage in the undesirable activity or to resist the urge to engage in the undesirable activity and ride out the craving. The determination whether it would be more beneficial for user 100 to mindfully engage in the undesirable activity or to resist the urge to engage in the undesirable activity and ride out the craving can be based on a prediction that the learning signal (αδt) will likely be a negative value.

The present technology aims to facilitate a learning process in which user 100 steadily learns that the undesirable behavior is less rewarding than believed, thus facilitating an end to partaking in the behavior. For any given craving, having user 100 partake in the undesirable behavior or ride out the craving may be the option which better achieves this goal. To best serve this purpose, application 115 can weigh the available data to encourage the best possible option based on the expected learning signal (αδt) user 100 will realize.

The method illustrated in FIG. 3B reflects and algorithm that identifies the best option (engage in the undesired activity, or ride out the activity) to reduce the reward value the user associates with the undesired activity. In FIG. 3B, application 115 determines (380) if user 100 has used the mindfulness exercise facilitated by application 115 via user interface 200 at least a threshold number of times. The threshold could be five times, 3 times, or using application 115 for over a week, as examples. This threshold aims to measure whether or not user 100 is sufficiently familiar with the mindfulness process, and/or whether there is sufficient data to make reasonable predictions about the current reward value the user associates with the undesired behavior. For instance, if user 100 has no used application 115 much, application 115 may not be able to provide a reasonable estimate of the reward value the user associates with the undesired behavior and more data is needed.

If user 100 has used the mindfulness exercise at least the threshold number of times, application 115 determines (382) if the most recent reward value (Vₜ) has sufficiently decreased since the user began using the application. This can be determined by analyzing data in a user profile associated with the user to approximate a starting reward value (V₁) of user 100 and to determine a relative reduction in reward value over time. An example threshold could be to determine that the current reward value that user 100 associates with the undesired behavior is less than 66% of the starting reward value. Another threshold, such as 50% of the starting reward value or a threshold measuring the average change in reward value per mindfulness exercise, can be used as well. This threshold aims to measure whether or not the past mindfulness exercises undertaken by user 100 have caused a meaningful drop in valuation of the undesirable behavior. A small change relative to the first reward value could indicate user 100 needs to go through the exercise of mindfully engaging in the behavior more times. This step attempts to estimate how capable user 100 might feel with respect to riding out the craving. If user 100 associates a reward value with the undesired behavior that is too similar to the starting reward value that existed when the user first sought help from the application, it may be too difficult for the user to attempt to ride out the craving.

If the most recent reward value of user 100 is not less than the threshold relative to the starting reward value, application 115 determines (384) if user 100 has used the mindfulness exercise facilitated by application 115 via user interface 200 at least a threshold number of times. The threshold can be use of application 115 8 times, 10 times, 12 times, 15 times, 20 times, or using application 115 for over a month. This threshold aims to measure whether user 100 has been using application 115 enough despite a relatively low reduction in reward value associated with the undesired behavior. It has been determined that most users that use application 115 between 10-15 times experience a significant reduction in reward value—even to near zero reward value. Accordingly, determination 384 attempts to compare the measured data against expected results. If the expected results (a significant reduction in reward value after using application 115 more than a threshold number of times) are not observed, application 115 can determine that user 100 should attempt to ride it out in order to allow user 100 to experience that they do not need to engage in the undesired behavior to overcome a craving, and this would be expected to result in a future reduction in reward value.

If the most recent reward value of user 100 is less than the threshold percent of the first reward value, or if the most recent reward value of user 100 is greater than the threshold percent of the first reward value but user 100 has used the mindfulness exercise at least ten times, application 115 determines (386) whether user 100 has received training for riding out the craving. Training can include a module on application 115, an independent training program to which application 115 has data access, or that user 100 has indicated they have received training on riding out cravings. Determination 386 is aimed at ensuring user 100 has the training necessary to successfully ride out the craving in a way that will result in a lower future reward value. For instance, if user 100 were instructed to ride out the craving but was not able to do so, that could affect the learning trajectory of user 100, the faith of user 100 in application 115, or further weaken the ability of user 100 to ride out future cravings.

If user 100 has training for riding out the craving, application 115 determines (388) if the craving score of user 100 is above some threshold. This threshold can be a set craving level, a function of current craving combined with past cravings, etc. This threshold aims to measure the intensity of the craving experienced by user 100, and for application 115 to factor that into its decision for whether user 100 should partake in the undesirable behavior. For instance, if user 100 had a relatively strong craving, a facilitated mindfulness exercise may demonstrate to user 100 a greater learning signal (resulting in a lower future reward value) than riding out the craving would.

If the craving score is under the threshold, application 115 determines (390) if the craving of user 100 is based on physiological need. Biometric data including glucose levels, stress indicators, brain activity, heart rate, body temperature, breathing rate, or skin conductance can be used to measure the state of user 100. This information aims to measure the need behind the craving experienced by user 100, and for application 115 to factor that into its decision for whether user 100 should partake in the undesirable behavior. For instance, low blood glucose levels could indicate that a craving for food is based on genuine hunger as opposed to a compulsive, undesirable behavior, and thus application 115 could encourage user 100 to partake in the undesirable behavior.

If all conditions and thresholds are satisfied in a satisfactory way, application 115 can encourage (394) user 100 to ride out the craving. This can entail different kinds of mindfulness exercises, the suggestion of an alternative task to the craving (for example, chewing gum instead of smoking), or something else entirely. If not all conditions and thresholds are satisfied, application 115 can facilitate (392) partaking in the undesirable behavior, similar to steps 330 through 370 shown in FIG. 3A.

The present technology details a software application that encourages behavior change through user interface responses. By encouraging a user to mindfully contemplate an undesirable activity and self-assess a craving score and subsequently refrain or mindfully partake in the undesirable activity and self-assess a contentedness score, the present technology facilitates the strengthening of mental habits which make it easier for a user to refrain from the undesirable activity in the future. These insights are supported by an ever-growing body of research.

The facilitation of mindfulness and the reception of craving and contentedness scores on their own can do a great deal to put users in a mindset better suited to combating an undesirable habit. However, the present technology can go further than facilitating these experiences when a user desires. By using a combination of data input by a user, insights from a population of users, results from ongoing research, biometric data, and other factors, the present technology can nudge a user's decisions and present mindful exercises without a user's prompting.

Using predictive algorithms, including regression models, machine learning models, or neural networks, the present technology can learn what actions a user should take at a given time or place to best lead to long-term refraining from the undesirable behavior. Using up-to-date research and open-source data from peer-reviewed papers, these algorithms can use a combination of research insights, past user data, and data from other users of the present technology to encourage better actions and activities.

In some embodiments, the user interface can be dynamically modified based on the inputs received by application 115. For example, when a user's craving score given after the mindfulness exercise is less than a threshold, user interface 200 shown in FIG. 2C can be dynamically modified to remove the option to partake in the activity, or make the button to select that option much less prominent than the button to skip the activity. In some embodiments, the user can be presented with a notification explaining that it is time for the user to attempt to ride out the craving, or to provide the user with an encouraging message or video.

For example, in some embodiments, the present technology can utilize data received by application 115 and that can be stored in a user profile on device 110 or server 120 to modify aspects of user interface 200. For example, analysis of a user profile can indicate that while a user's craving score given after the mindfulness exercise often indicates less of a craving than the user experienced prior to the mindfulness exercise, the user often decides to partake in the habitual activity. In response to this determination, user interface 200 shown in FIG. 2C can be dynamically modified to remove the option to partake in the activity, or make the button to select that option much less prominent than the button to skip the activity. In some embodiments, the user can be presented with a notification explaining that it is time for the user to attempt to ride out the craving, or to provide the user with an encouraging message or video.

In another example, analysis of data in the user profile can show that in the past when a user gave a lower relative craving score, but still engaged in the habitual activity that the user often gave a low contentedness score after engaging in the habitual activity under those circumstances. Based on such analysis, when a user's craving score input in an interface such as shown in FIG. 2C indicates that the craving is less than the user experienced prior to the mindfulness exercise, application 215 can provide a notification reminding a user that they gave a poor contentedness score the last time they engaged in the habitual behavior under a similar circumstance.

Biometric data can provide a particularly rich insight into a user's current condition. Brain wave data from an electroencephalogram (EEG) can indicate how far a user may be from a mindful state, given the neural signatures of mindfulness. Heart rate can indicate a user's current anxiety or stress level, along with skin conductance or cortisol levels. Pupillometry can indicate levels of arousal. While not all embodiments of the present technology will have access to these data, either in the present or from past biometrics, when available they can provide a rich input to a predictive algorithm.

These predictions can then be used to, for instance, encourage a user to partake or refrain from the undesirable activity, alter or skip the mindfulness exercises that precede measures of craving or contentedness, encourage a user to check-in, or begin any of these activities without user prompting. Predictions and suggestions can also be based on well-established psychological models of decision making, such as the Rescorla-Wagner model, to determine how close the user might be to partaking in the undesirable activity or refraining from it. In some instances, this data may even be plotted and presented to the user as a means to visually demonstrate progress towards the ultimate goal.

In some embodiments, biometric data can be stored in a user profile and analyzed with respect to other data provided manually into the application 115. For example, biometric data can be analyzed for periods preceding the user opening application 115. Biometric data accumulated during this period can be include signals of craving. Biometric data can also be analyzed for periods during and after a user reports partaking in the unwanted behavior. Such analysis can identify markers that might indicate a craving is coming or passing. Application 115 can monitor biometric data reading in real time to identify and predict when a craving is about to hit and can use this information to trigger an intervention to help the user be mindful throughout the craving period.

When application 115 predicts a craving might be coming on, it can also provide a notification to the user to suggest that the user engage in an alternate activity, like to take a walk. Some habitual behaviors can be triggered by situations, and application 115 can predict that a craving might be coming on and encourage the user to remove themselves from the triggering situation.

In some embodiments, application 115 can analyze user profile data to identify milestones along a path toward behavior change and reward uses with statements of praise or awards. In some embodiments, milestones can be tied to changes in a user's Rescorla-Wagner score that measure the strength of the relationship between the craving score and the contentedness score. As this relationship decreases, it may be considered that the user is closer to achieving the behavior change. Progress in this measure can be tied to milestones at which a user should receive encouragement.

Through a combination of user interface inputs and mindfulness exercises, the present technology serves to change users thought patterns and ultimately their behaviors. Backed by a growing literature, this technology serves a need in the art.

In addition to helping a user refrain from undesirable behaviors, the present technology can be used to help a user foster desirable behaviors. In one embodiment, user 100 can decide to make a habit of going for a run every morning. Application 115 can remind user 100 at an appropriate time every morning, prompting user 100 to complete a mindfulness exercise contemplating the experience and benefits of running. After completing the mindfulness exercise, application 115 can present a slider to receive a desire score (analogous to a craving score in the undesirable behavior embodiments). In this case, as with other embodiments dealing with desirable behaviors, the mindfulness exercise is intended to increase the desire of user 100 to partake in the behavior, as opposed to decreasing it. User 100 can then decide whether to engage in running or not, and, after running mindfully, input a contentedness score into the interface presented by application 115. The above describes only one embodiment dealing with desirable behaviors. As with undesirable behaviors, application 115 can use predictive algorithms, biometrics, past data from user 100, past data from other users, or other factors to optimally guide user 100 in the journey to form a habit around the desirable behavior. Application 115 can passively receive inputs from user 100, or can prompt user 100 at certain times.

Thus, more broadly, the present technology provides a technological framework for fostering or reducing behaviors. After presenting a mindfulness exercise to a user, the application is configured to receive a first score from the user, which can be either a craving score for an undesirable behavior or a desire score for a desirable behavior. This first score measures the current state of mind of the user that takes into account mental, emotional, and physical states after the mindfulness exercise. After engaging or not engaging in the behavior (mindfully), the application is configured to receive a contentedness score, indicating the current contentedness of the user.

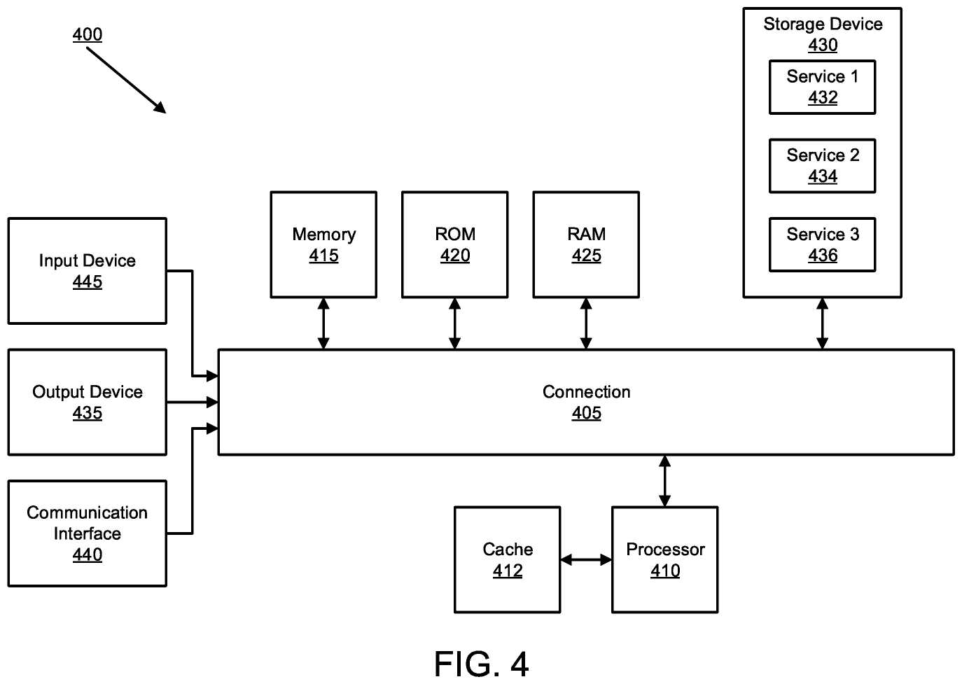

FIG. 4 shows an example of computing system 400, which can be for example any computing device making up device 110 or server 120 or any component thereof in which the components of the system are in communication with each other using connection 405. Connection 405 can be a physical connection via a bus, or a direct connection into processor 410, such as in a chipset architecture. Connection 405 can also be a virtual connection, networked connection, or logical connection.

In some embodiments computing system 400 is a distributed system in which the functions described in this disclosure can be distributed within a datacenter, multiple datacenters, a peer network, etc. In some embodiments, one or more of the described system components represents many such components each performing some or all of the function for which the component is described. In some embodiments, the components can be physical or virtual devices.

Example system 400 includes at least one processing unit (CPU or processor) 410 and connection 405 that couples various system components including system memory 415, such as read only memory (ROM) 420 and random access memory (RAM) 425 to processor 410. Computing system 400 can include a cache of high-speed memory 412 connected directly with, in close proximity to, or integrated as part of processor 410.

Processor 410 can include any general purpose processor and a hardware service or software service, such as services 432, 434, and 436 stored in storage device 430, configured to control processor 410 as well as a special-purpose processor where software instructions are incorporated into the actual processor design. Processor 410 may essentially be a completely self-contained computing system, containing multiple cores or processors, a bus, memory controller, cache, etc. A multi-core processor may be symmetric or asymmetric.

To enable user interaction, computing system 400 includes an input device 445, which can represent any number of input mechanisms, such as a microphone for speech, a touch-sensitive screen for gesture or graphical input, keyboard, mouse, motion input, speech, etc. Computing system 400 can also include output device 435, which can be one or more of a number of output mechanisms known to those of skill in the art. In some instances, multimodal systems can enable a user to provide multiple types of input/output to communicate with computing system 400. Computing system 400 can include communications interface 440, which can generally govern and manage the user input and system output. There is no restriction on operating on any particular hardware arrangement and therefore the basic features here may easily be substituted for improved hardware or firmware arrangements as they are developed.

Storage device 430 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read only memory (ROM), and/or some combination of these devices.

The storage device 430 can include software services, servers, services, etc., that when the code that defines such software is executed by the processor 410, it causes the system to perform a function. In some embodiments, a hardware service that performs a particular function can include the software component stored in a computer-readable medium in connection with the necessary hardware components, such as processor 410, connection 405, output device 435, etc., to carry out the function.

For clarity of explanation, in some instances the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program, or a collection of programs that carry out a specific function. In some embodiments, a service can be considered a server. The memory can be a non-transitory computer-readable medium.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, solid state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

1. A non-transitory computer readable medium comprising instructions stored thereon, the instructions effective to cause at least one processor to:
present, by an application, information in a user interface instructing a user to think about an aspect of a craving to partake in an undesired behavior;
after the presentation of the user interface of the information instructing the user to think about the aspect of the craving to partake in the undesired behavior, present an user interface element that is configured to receive a craving score that is an indication of how the craving to partake in the undesired behavior has changed relative to a time prior to the presentation of the information instructing the user to think about the aspect of the craving to partake in the undesired behavior;
receive the craving score in the user interface; and
based on the craving score, and the number of times the user has been presented with the information instructing the user to think about the aspect of the craving to partake in the undesired behavior, adjust a variable that effects a presentation of options, wherein one option is to ignore the craving and another option is to partake in the behavior.

2. The non-transitory computer readable medium of claim 1 comprising further instructions effective to cause the at least one processor to:
after the receipt of the craving score in the user interface, determine, by the application, whether to encourage the user to ignore the craving to partake in the undesired behavior or to encourage the user to mindfully partake in the undesired behavior.

3. The non-transitory computer readable medium of claim 1 comprising further instructions effective to cause the at least one processor to:
receive an input that indicates the user would like to mindfully partake in the undesired behavior;
after the receipt of the input that indicates that the user would like to mindfully partake in the undesired behavior, present an interface that is configured to receive a contentedness score that is an indication of the contentedness of the user after partaking the undesired behavior.

4. (canceled)

5. The non-transitory computer readable medium of claim 4, wherein the variable is a size, color, and/or animation of a selectable button associated with at least one of the options.

6. The non-transitory computer readable medium of claim 3 comprising further instructions effective to cause the at least one processor to:
store the craving score and the contentedness score with a time stamp in a user profile;
generate, using a set of past craving scores, contentedness scores, and time stamps, a Rescorla-Wagner model to measure the strength of the relationship between the craving score and the contentedness score.

7. The non-transitory computer readable medium of claim 3 comprising further instructions effective to cause the at least one processor to:
store the craving score and the contentedness score with a time stamp in a user profile;
based on data stored in associated with the user profile, predict that a craving might occur; and
present a suggestion to the user to engage in an activity the might prevent the craving.

8. The non-transitory computer readable medium of claim 3 comprising further instructions effective to cause the at least one processor to:
monitor biometric data of the user during any of: the presentation information instructing a user to think about an aspect of a craving to partake an undesired behavior, or the period in which the user is partaking in the undesired activity;
correlate the biometric data with the craving score and the contentedness score to identify outlier craving score entry or contentedness score entry.

9. The non-transitory computer readable medium of claim 1 comprising further instructions effective to cause the at least one processor to:
monitor biometric data of the user over time;
based on the biometric data predict a craving might occur; and
present a suggestion to the user to engage in an activity that might prevent the craving.

10. A method comprising:
presenting, by an application, information in a user interface instructing a user to think about an aspect of a behavior;
after the presentation of the information instructing the user to think about the aspect of the behavior, presenting an user interface element that is configured to receive a first score that is an indication of how the state of mind of the user pertaining to the behavior has changed relative to a time prior to the presentation of the information instructing the user to think about the aspect of the behavior;
receiving the first score in the user interface; and
based on the first score, and the number of times the user has been presented with the information instructing the user to think about the aspect of the behavior, adjusting a variable that effects a presentation of options, wherein one option is to ignore the craving and another option is to partake in the behavior.

11. The method of claim 10 further comprising:
after the receipt of the first score in the user interface, determining, by the application, whether to encourage the user to ignore a craving to partake in the behavior or to encourage the user to mindfully partake in the behavior.

12. The method of claim 10 further comprising:
receiving an input that indicates the user would like to mindfully partake in the behavior;
after the receipt of the input that indicates that the user would like to mindfully partake in the behavior, presenting an interface that is configured to receive a contentedness score that is an indication of the contentedness of the user after partaking the behavior.

13. (canceled)

14. The method of claim 13, wherein the variable is a size, color, and/or animation of a selectable button associated with at least one of the options.

15. The method of claim 12 further comprising:
storing the first score and the contentedness score with a time stamp in a user profile;
generating, using a set of past first scores, contentedness scores, and time stamps, a Rescorla-Wagner model value to measure the strength of the relationship between the first score and the contentedness score.

16. The method of claim 10 further comprising:
monitoring biometric data of the user over time;
based on the biometric data predicting a craving might occur; and
presenting a suggestion to the user to engage in an activity that might prevent the craving.

17. A system comprising:
at least one non-transitory computer readable medium having instructions stored thereon; and
at least one processor, the instructions effective to cause the at least one processor to:
present, by an application, information in a user interface instructing a user to think about an aspect of a craving to partake in an undesired behavior;
after the presentation of the user interface of the information instructing the user to think about the aspect of the craving to partake in the undesired behavior, present an user interface element that is configured to receive a craving score that is an indication of how the craving to partake in the undesired behavior has changed relative to a time prior to the presentation of the information instructing the user to think about the aspect of the craving to partake in the undesired behavior;
receive the craving score in the user interface; and
based on the craving score, and the number of times the user has been presented with the information instructing the user to think about the aspect of the craving to partake in the undesired behavior, adjust a variable that effects a presentation of options, wherein one option is to ignore the craving and another option is to partake in the behavior.

18. The system of claim 17 wherein the instructions are effective to cause the at least one processor to:
after the receipt of the craving score in the user interface, determine, by the application, whether to encourage the user to ignore the craving to partake in the undesired behavior or to encourage the user to mindfully partake in the undesired behavior.

19. The system of claim 17 wherein the instructions are effective to cause the at least one processor to:
receive an input that indicates the user would like to mindfully partake in the undesired behavior;
after the receipt of the input that indicates that the user would like to mindfully partake in the undesired behavior, present an interface that is configured to receive a contentedness score that is an indication of the contentedness of the user after partaking the undesired behavior.

20. (canceled)

21. The method of claim 12 further comprising:
storing the first score and the contentedness score with a time stamp in a user profile;
based on data stored in associated with the user profile, predicting that a craving might occur; and
presenting a suggestion to the user to engage in an activity that might prevent the craving.

22. The method of claim 12 further comprising:
monitoring biometric data of the user during any of: the presentation information instructing a user to think about an aspect of the behavior, or the period in which the user is partaking in the undesired activity;
correlating the biometric data with the first score and the contentedness score to identify outlier craving score entry or contentedness score entry.
